(19) United States
(12) Patent Application Publication
(10) Pub. No.: US 2025/0032494 A1
(43) Pub. Date: Jan. 30, 2025

(54) A COMBINATION TREATMENT FOR CANCER

(71) Applicant: My Personal Therapeutics LTD, London (GB)

(72) Inventors: Santiago Nahuel Villegas, London (GB); Laura Towart, London (GB)

(21) Appl. No.: 18/716,737

(22) PCT Filed: Dec. 5, 2022
(86) PCT No.: PCT/IB2022/000744

§ 371 Date: Jun. 5, 2024

Related U.S. Application Data
(60) Provisional application No. 63/286,255, filed on Dec. 6, 2021.

(51) Int. Cl. A61K 31/519; A61K 31/4035

(52) U.S. Cl. CPC A61K 31/519; A61K 31/4035

(57) ABSTRACT

The invention recognizes a combination of drugs that is effective against cancers that exhibit a KRAS mutation. The invention provides a combination therapy that includes the two agents, trametinib and apremilast for the treatment of certain cancers.

### FIELD OF THE INVENTION

The invention relates to the treatment of cancer with a combination of non-cytotoxic agents, trametinib and apremilast.

### BACKGROUND

Cancer is a major driver of morbidity, mortality, and health care costs around the world. Traditionally, cancers have been treated with broad spectrum cytotoxic agents that have grave and widespread side effects. More recently, some cancers have been found to be easily targeted by more specific novel agents including monoclonal antibodies and kinase inhibitors that have fewer adverse effects than cytotoxic therapy. However, there are still other malignancies caused by mutations to proteins that are more difficult to target.

KRAS is a gene that has known oncogenic mutations. KRAS has been linked to lung, colorectal, and pancreatic cancers. The protein encoded by the KRAS gene is small and has a smooth surface with no potential binding sites, which means that directed pharmaceutical treatment of KRAS-mutation cancers has proven elusive.

### SUMMARY

The invention recognizes a combination of drugs that is effective against cancers that exhibit a KRAS mutation. To that end, in certain aspects, the invention provides a combination therapy for treatment of certain cancers, the therapy including the two agents, trametinib and apremilast (including optionally analogs, derivatives, prodrugs, or pharmaceutically acceptable salts thereof). Trametinib is a kinase inhibitor that targets the MEK1 and MEK2 protein kinase enzymes; this results in decreased cell signaling for cell division and proliferation and may have effect on reducing tumor growth in this way. Apremilast is a phosphodiesterase-4 (PDE4) inhibitor that also inhibits production of Tumor Necrosis Factor-alpha (TNF-α). TNF-α participates in the inflammatory pathway, leading to the use of the drug in autoimmune disorders where the body's inflammatory process is hyperactive.

According to an embodiment of the invention, the combination can be administered orally, intravenously, or subcutaneously. Advantageously, both agents are well-absorbed orally. The combination therapy may include one dosing regimen for one of the agents, such as once a day, twice a day, three times a day, four times a day, or more or less frequently; the other agent may be administered on a separate schedule once a day, twice a day, three times a day, four times a day, or more or less frequently.

In some embodiments, the combination includes both agents being administered in a single dosage form. In other embodiments, the combination may be administered separately. In embodiments in which both agents are contained in the same dosage form, the agents may be provided in an even ratio of both agents. In other embodiments, the agents are administered in an uneven ratio, such as 2:1, 3:1, 4:1, 5:1, 6:1, 7:1, 8:1, 9:1, 10:1, 11:1, 12:1, 13:1, 14:1, 15:1, or a greater or lesser relative concentration.

In other embodiments of the invention, the combination is provided in a sustained release formulation. The sustained release may be provided by a matrix, osmotically releasing oral system, enteric coated tablet, other coated tablet, or capsule, by a tablet contained within a capsule, or by any other pharmaceutically convenient time control mechanism, whether considered controlled release, sustained release, extended release, delayed release, or another time control mechanism. One or both agents may be provided in such a time release mechanism.

In an embodiment of the invention, due consideration is given to patient specific factors such as prior treatment for cancer, tolerability of the treatment, or drug-disease interactions such as renal impairment. For one or all those considerations, the dose of one or both agents may be reduced to account for the patient-specific factors. For example, the dose could be reduced 25%, 50%, or 75%, or greater or lesser dosages, or the duration between doses could be increased by 25%, 50%, 100%, 200%, or greater or lesser frequency dosing schedules.

Advantageously, embodiments of the invention may be effective against cancers that are related to a mutation in the KRAS gene, and the protein thereby encoded. KRAS mutations have been detected in lung, colorectal, and pancreatic cancers. In other embodiments of the invention, the subject's genotype has not yet been determined and the combination treatment is administered to a subject regardless of the genotype of the mutations in the cancer for which the subject is being treated.

In another embodiment of the invention, the invention is a method of treating malignancies by administering the combination treatment to a subject. The method may include any of the above modifications to the combination, any of the dosage forms of the combination, and any of the possible ratios or plans of administration of both or either of the agents.

### DETAILED DESCRIPTION

The invention provides combination therapies for treatment of malignancy in a subject, the combination therapy comprising trametinib and apremilast, or analogs, derivatives, prodrugs, or pharmaceutically acceptable salts thereof.

Malignancies, or cancers, are a vast and diverse group of diseases that involve uncontrolled cell growth. The malignancy need not cause a subject any noticeable symptoms, or the subject may be experiencing debilitating signs and symptoms of disease. Malignancies can affect any tissue or organ system of the subject, such as skin, blood, lymph, gastrointestinal, musculoskeletal, genitourinary, ocular, auditory, oral, nasopharyngeal, respiratory, and cardiovascular tissues, organs, or organ systems.

Malignancies may have varying genetic mutations that cause the pathogenesis of that cancer. Advantageously, some embodiments have increased activity against cancers having the KRAS genetic mutation. In healthy cells, KRAS serves as an on-off switch that regulates cell growth. It does this by binding a KRAS-activating molecule called GTP and then converts it to GDP, which inactivates the protein. However, when the gene is mutated, KRAS can become permanently activated, allowing cells to grow uncontrollably and to activate downstream pathways. This leads to multiplication of cells and cancer growth, which can then cause metastases. KRAS mutations are present in approximately 25% of tumors, making them one of the most common gene mutations linked to cancer. They are frequent drivers in lung, colorectal and pancreatic cancers. KRAS drives 32% of lung cancers, 40% of colorectal cancers, and 80% to 85% of pancreatic cancer cases.

The protein encoded by KRAS has long been considered an “undruggable” target because it is a small protein with very limited potential binding sites. Advantageously, embodiments of the invention may be effective at treating KRAS-mutation positive cancers. The effect of the combination treatment on such malignancies may derive from the synergistic effects of the combination on the pathways activated by the mutated KRAS.

Embodiments of the invention encompass the administration of both trametinib and apremilast to a subject (for example in a single dosage form or as divided dosages). In divided dosage embodiments, the two drugs may be administered on the same dosing schedule or according to their own dosing schedules. The dosage of the combination of trametinib and apremilast may be provided separately in discrete dosage forms, or the dosages may be provided in a fixed dose combination single dosage form. In embodiments where both agents are contained in the same dosage form, the agents may be provided in an even ratio of both agents. In other embodiments, the agents are administered in an uneven ratio, such as 2:1, 3:1, 4:1, 5:1, 6:1, 7:1, 8:1, 9:1, 10:1, 11:1, 12:1, 13:1, 14:1, 15:1, or a greater or lesser relative concentration. Determination of the dosage and formulation of the dosage form follows.

### Trametinib

Trametinib is a kinase inhibitor drug that has demonstrated anti-cancer activity. It inhibits the mitogen-activated protein kinase kinase enzymes MEK1 and MEK2. MEK1 and MEK2 participate in an intracellular signaling pathway known as the Ras-Raf-MEK-ERK pathway. This pathway communicates from cell surface proteins into the nucleus of a cell to activate or deactivate cell division. Breakdown of the pathway due to mutations in one of the genes that encode any of the proteins involved in the pathway can be oncogenic. MEK1 and MEK2 are involved in proliferation, differentiation, transcription regulation and cellular development. Trametinib's inhibition of this pathway can reduce oncological growth. Trametinib inhibits cell growth of tumors with the BRAF V600 mutation both in vitro and in vivo.

Trametinib has the following structure.

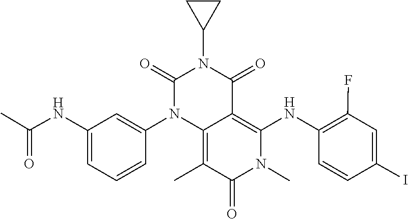 [SMILES: CC(=O)Nc1cccc(-n2c(=O)n(C3CC3)c(=O)c3c(Nc4ccc(I)cc4F)n(C)c(=O)c(C)c32)c1]

Trametinib was originally studied in an international, multicenter, randomized, open-label, active-controlled trial. Trametinib was evaluate against cytotoxic active controls, either dacarbazine, or paclitaxel. That trial resulted in statistically significant increase in progression-free survival in the patients treated with trametinib. Similar trials and results were found when trametinib was studied in combination with dabrafenib, in both dabrafenib-controlled and placebo-controlled trials.

Trametinib has been approved for administration to cancer patients alone and in combination with other medications. Trametinib has been approved alone or in combination for various cancers including melanoma, lung cancer, and thyroid cancer. Trametinib can be administered orally at up to 2 mg daily for approved indications, and the dose may be reduced for adverse effects. Trametinib may cause side effects including hyperglycemia, fetal toxicity, clotting disorders, hemorrhage, ocular toxicity, pulmonary symptoms, skin toxicity, colitis and gastrointestinal perforation, and serious fever. Trametinib is administered continuously until treatment success, progression, or toxicity occurs. Trametinib is commercially available as 0.5 and 2 mg tablets.

Trametinib is described for example in the following publications, which are hereby incorporated by reference in their entirety for all purposes: U.S. Pat. Nos. 7,378,423, 8,580,304, 8,703,781, 8,835,443, 8,952,018, 9,155,706, 9,271,941, 9,399,021, and 10,869,869.

### Apremilast

Apremilast is phosphodiesterase-4 (PDE4) inhibitor that also inhibits production of Tumor Necrosis Factor-alpha (TNF-α). As a PDE4 inhibitor, apremilast demonstrates activity by reducing the breakdown of cyclic adenosine monophosphate (cAMP). This decreases the expression of inflammatory markers such as TNF-α, interleukin 17, interleukin 23, and others. The decrease in TNF-α production is responsible for apremilast being used primarily as a treatment for autoimmune disorders where TNF-α production results in inflammatory morbidities.

Apremilast has the following structure.

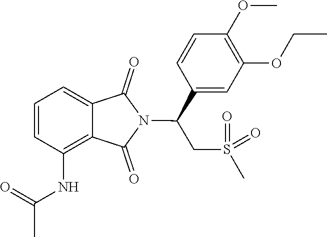 [SMILES: CCOc1cc([C@@H](CS(C)(=O)=O)N2C(=O)c3cccc(NC(C)=O)c3C2=O)ccc1OC]

Apremilast was originally studied in multicenter, randomized, double-blind, placebo-controlled trials with and without co-administration of traditional psoriatic arthritis treatment, typically known as disease-modifying anti-rheumatoid drugs (DMARDs). The DMARDs studied within the trial were methotrexate, sulfasalazine, leflunomide, oral steroids, and nonsteroidal anti-inflammatory drugs. Apremilast yielded statistically significant improvement over placebo in patients. Apremilast was also studied in patients with plaque psoriasis without arthritis, and patients with oral lesions associated with Behçet's disease.

Apremilast has been approved for administration to patients with psoriasis, psoriatic arthritis, and Behçet's disease. Apremilast must be titrated upward at the initiation of therapy. The dose may be reduced for renal impairment. Apremilast may cause adverse effects, such as depression, weight loss, diarrhea, nausea, upper respiratory tract infection, and headache. Apremilast may be administered twice daily or less often in renal impairment. Apremilast is administered continuously for an indefinite period; there is no limitation on treatment.

Apremilast is described for example in the following publications, which are hereby incorporated by reference in their entirety for all purposes: U.S. Pat. Nos. 6,962,940, 7,208,516, 7,427,638, 7,659,302, 7,893,101, 8,455,536, 8,802,717, 9,018,243, 9,724,330, 9,872,854, and 10,092,541.

### Determination of Dosage

The dosing regimen may include a dose, i.e., an amount, of the agents trametinib and apremilast that should be administered. The dosing regimen may include a time point for administration of a dose of the agents to the subject. The dosing regimen may be based on one or more measured levels of the agents in a sample obtained from the subject, which allows the dosing regimen to be tailored to an individual subject, e.g., a patient.

Consequently, the methods of the invention provide customized dosing regimens that account for variability in pharmacokinetic properties, i.e., metabolism of the active pharmaceutical ingredient (API) by the subject, and pharmacodynamics properties, effect of the API on its target, among individuals. The dosing regimen may be determined by comparing a measured level of the agents in a sample obtained from a subject to a reference that provides an association between the measured level and a recommended dosage adjustment of the agents. For example, the reference may provide a relationship between administration of the agents and levels of either agent in the subject. The relationship can be empirically determined from a known dose and time of administration of the agents and measured levels of either agent at one or more subsequent time points. The reference may include a relationship between measured levels of the agents or a metabolic product of the agents and measured levels of either agent.

From the comparison between the measured level of the agents trametinib and apremilast and the reference, a dosing regimen may then be determined. The dosing regimen may include a dosage of the agents, a time for administration of the dosage, or both. The dosing regimen may be determined de novo, or it may comprise an adjustment to a previous dosing regimen, such as an adjustment in the dosage, the interval between administration of dosages, or both. The dosing regimen is designed to deliver the agents to the subject in an amount that achieves a therapeutic effect. The therapeutic effect may be a sign or symptom of a disease, disorder, or condition. The therapeutic effect may be inhibition of an enzyme in the metabolic pathway, or it may be a change in an indicator of inhibition of an enzyme in a metabolic pathway. The therapeutic effect may be a decrease in number of cancer cells, a decrease in proliferation of cancer cells, an increase in differentiation of pre-cancerous cells, such as myeloblasts, complete remission of cancer, complete remission with incomplete hematologic recovery, morphologic cancer-free status, or partial remission.

The dosing regimen may ensure that levels of the agents trametinib and apremilast are raised or maintained above a minimum threshold required to achieve a certain effect. For example, the dosing regimen may raise or maintain levels of the agents above a threshold level in the subject for a certain time period. The time period may include a minimum, a maximum, or both. For example, the dosing regimen may raise or maintain levels of the agents above the threshold level for at least 6 hours, 12, hours, 24 hours, at least 48 hours, at least 72 hours, at least 84 hours, at least 96 hours, at least 5 days, at least 6 days, at least 7 days, at least 10 days, at least 2 weeks, or more. The dosing regimen may raise or maintain levels of the agents above the threshold level for not more than 24 hours, not more than 36 hours, not more than 48 hours, not more than 60 hours, not more than 72 hours, not more than 84 hours, not more than 96 hours, not more than 5 days, not more than 6 days, not more than 7 days, not more than 10 days, or not more than 2 weeks. The dosing regimen may raise or maintain levels of the agents above the threshold level for at least 72 hours but not more than 96 hours, for at least 72 hours but not more than 5 days, for at least 72 hours but not more than 6 days, for at least 72 hours but not more than 7 days, for at least 96 hours but not more than 7 days.

The dosing regimen may ensure that levels of the agents trametinib and apremilast do not exceed or are maintained below a maximum threshold that is associated with toxicity. Levels of the agents above a maximum threshold may indicate that the agents are causing or are likely to cause an adverse event in the subject. For example and without limitation, adverse events include abdominal pain, anemia, anorexia, blood disorder, constipation, diarrhea, dyspepsia, fatigue, fever, granulocytopenia, headache, infection, leukopenia, mucositis, nausea, pain at the injection site, phlebitis, photosensitivity, rash, somnolence, stomatitis, thrombocytopenia, and vomiting.

The dosing regimen may include a time point for administration of one or more subsequent doses to raise or maintain levels of the agents above a minimum threshold level for a certain time period. The time point for administration of a subsequent dose may be relative to an earlier time point. For example, the time point for administration of a subsequent dose may be relative to a time point when a previous dose was administered or a time point when a sample was obtained from a subject.

The dosing regimen may include a schedule for administration of doses. For example, doses may be administered at regular intervals, such as every 6 hours, every 12 hours, every 24 hours, every 36 hours, every 48 hours, every 60 hours, every 72 hours, every 84 hours, every 96 hours, every 5 days, every 6 days, every week, every 2 weeks, every 3 weeks, or every 4 weeks. Alternatively, doses may be administered according to a schedule that does not require precisely regular intervals. For example, doses may be administered once per week, twice per week, three times per week, four times per week, once per month, twice per month, three times per month, four times per month, five times per month, or six times per month.

For example and without limitation, an oral dosing regimen for administration of trametinib to a human subject may be as follows: 0.5 mg daily, 1 mg daily, 1.5 mg daily, 2 mg daily, 0.5 mg every other day, 1 mg every other day, 1.5 mg every other day, or 2 mg every other day.

For example and without limitation, an oral dosing regimen for administration of apremilast to a human subject may be as follows: 10 mg twice daily, 10 mg in the morning and 20 mg in the evening, 20 mg twice daily, 20 mg in the morning and 30 mg in the evening, 30 mg twice daily, 10 mg daily, 20 mg daily, 30 mg daily, 10 mg every other day, 20 mg every other day, or 30 mg every other day.

Dosing of the trametinib and apremilast may account for the formulation of trametinib and apremilast. For example, trametinib and apremilast may be provided as prodrugs, analogs, derivatives, or salts. Any of the aforementioned chemical forms may be provided in a pharmaceutically acceptable formulation, such as a micellar formulation or sustained release formulation. Dosage of trametinib and apremilast also depends on factors such as the type of subject and route of administration. The dosage may fall within a range for a given type of subject and route of administration, or the dosage may be adjusted by a specified amount for a given type of subject and route of administration. Subject-specific factors such as renal impairment, adverse effects, or sensitivity reactions to the agent may cause dosage adjustment.

### Pharmaceutically Acceptable Carriers and Auxiliary Ingredients and Agents

In embodiments of the invention, the pharmaceutical formulation containing an amount of one or more of the agents trametinib and apremilast described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction of the agents described herein. The oral dosage form can be administered to a subject in need thereof. Where appropriate, the dosage forms described herein can be microencapsulated.

The dosage form can also be prepared to prolong or sustain the release of any ingredient. In some embodiments, the agents trametinib and apremilast can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the trametinib or apremilast is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the trametinib or apremilast and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of the trametinib or apremilast. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the trametinib and apremilast, an auxiliary active ingredient, and/or pharmaceutically acceptable salt thereof, such a dosage form can contain a powder base such as lactose, glucose, trehalose, mannitol, and/or starch. In some of these embodiments, the trametinib or apremilast is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain antioxidants, buffers, bacteriostats, solutes that render the composition isotonic with the eye or fluid contained therein or around the eye of the subject, and aqueous and nonaqueous sterile suspensions, which can include suspending agents and thickening agents.

### Examples

According to an embodiment of the invention, the activity of the trametinib and apremilast of the pharmaceutical combination, amongst other tested agents, have activity against target cancers according to the following tables.

TABLE 1
2-drug combinations having been screened in a subject with
a gastrointestinal malignancy, specifically pseudomyxoma peritonei.
The subject had KRAS and GNAS oncogenes present in the malignancy.
Asterisks indicate the strength of the screened effect on the malignancy.

| | 1rst drug | 2nd drug | effect |
|---|---|---|---|
| 1 | Trametinib+ | Apremilast | **** |
| 2 | Trametinib+ | Belinostat | **** |
| 3 | Trametinib+ | Rapamycin | **** |
| 4 | Trametinib+ | Zoledronic Acid | *** |
| 5 | Trametinib+ | Imatinib | *** |
| 6 | Combo 3 | Evero + PD03 | *** |
| 7 | Trametinib+ | Everolimus | *** |
| 8 | Trametinib+ | Fluvastatin | *** |
| 9 | Trametinib+ | Busulfan | *** |
| 10 | Trametinib+ | Afatinib | *** |
| 11 | Trametinib+ | Osimertinib | *** |
| 12 | Trametinib+ | Brigatinib | *** |
| 13 | Trametinib+ | Ponatinib | *** |
| 14 | Trametinib+ | Fluoxetine | *** |
| 15 | Trametinib+ | Ramelteon | *** |
| 16 | Trametinib+ | Flibanserin | ** |
| 17 | Trametinib+ | Temsirolimus | ** |
| 18 | Trametinib+ | Tirofiban | ** |

TABLE 2
3-drug combinations having been screened in a subject with a
gastrointestinal malignancy, specifically pseudomyxoma peritonei.
The subject had KRAS and GNAS oncogenes present in the malignancy.
Asterisks indicate the strength of the screened effect on the malignancy.

| Combos | drug1 | drug2 | drug3 | effect |
|---|---|---|---|---|
| Combo7 | Trametinib | Everolimus | Apremilast | **** |
| Combo8 | Trametinib | Everolimus | Tirofiban | **** |
| Combo9 | Trametinib | Everolimus | Fluvastatin | *** |
| Combo10 | Trametinib | Rapamycin | Apremilast | *** |
| Combo13 | Trametinib | Everolimus | Ramelteon | *** |
| Combo14 | Trametinib | Everolimus | Ivacaftor | ** |
| Combo16 | Trametinib | Rapamycin | Ivacaftor | ** |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A combination therapy for treatment of malignancy in a subject, the combination therapy comprising trametinib and apremilast, or analogs, derivatives, prodrugs, or pharmaceutically acceptable salts thereof.

2. The combination therapy of claim 1, wherein trametinib and apremilast are provided orally, intravenously, or subcutaneously.

3. The combination therapy of claim 1, wherein trametinib and apremilast are provided according to different dosing regimens.

4. The combination therapy of claim 1, wherein trametinib and apremilast are provided as a single dosage form.

5. The combination therapy of claim 1, wherein trametinib and apremilast are provided in divided dosage forms.

6. The combination therapy of claim 1, wherein trametinib and apremilast are provided in a fixed-dose combination dosage form.

7. The combination therapy of claim 1, wherein trametinib and apremilast are provided in a 1:1 ratio in a dosage form.

8. The combination therapy of claim 1, wherein trametinib and apremilast are provided in an unequal ratio in a dosage form.

9. The combination therapy of claim 1, wherein trametinib and apremilast are provided in a sustained release formulation.

10. The combination therapy of claim 1, wherein trametinib and apremilast are provided in reduced doses for subjects having renal impairment.

11. The combination therapy of claim 1, wherein the malignancy treated is a cancer having a KRAS mutation.

12. A method of treating malignancy in a subject, the method comprising providing to a subject having cancer a combination therapy comprising trametinib and apremilast, or analogs, derivatives, prodrugs, or pharmaceutically acceptable salts thereof.

13. The method of claim 13, wherein trametinib and apremilast are provided orally, intravenously, or subcutaneously.

14. The method of claim 13, wherein trametinib and apremilast are provided according to different dosing regimens.

15. The method of claim 13, wherein trametinib and apremilast are provided as a single dosage form.

16. The method of claim 13, wherein trametinib and apremilast are provided in divided dosage forms.

17. The method of claim 13, wherein trametinib and apremilast are provided in a fixed-dose combination dosage form.

18. The method of claim 13, wherein trametinib and apremilast are provided in a 1:1 ratio in a dosage form.

19. The method of claim 13, wherein trametinib and apremilast are provided in an unequal ratio in a dosage form.

20. The method of claim 13, wherein trametinib and apremilast are provided in a sustained release formulation.

21. The method of claim 13, wherein trametinib and apremilast are provided in reduced doses for subjects having renal impairment.

22. The method of claim 13, wherein the malignancy treated is a cancer having a KRAS mutation.
